(19) United States
(12) Patent Application Publication
(10) Pub. No.: US 2012/0142598 A1
(43) Pub. Date: Jun. 7, 2012

(54) EDIBLE COMPOSITIONS FOR PREVENTING HAIR LOSS

(76) Inventor: Zeev Pam, Misgav Dov (IL)

(21) Appl. No.: 12/745,931

(22) PCT Filed: Dec. 3, 2007
(86) PCT No.: PCT/IL2007/001490

§ 371 Date: Jun. 3, 2010

Related U.S. Application Data
(60) Provisional application No. 60/870,458, filed on Dec. 18, 2006.

(51) Int. Cl. A61K 38/39; A61P 17/14; C07K 14/78

(52) U.S. Cl. 514/17.2; 530/354

(57) ABSTRACT

The present invention provides compositions comprising hydrolyzed gelatin or a mixture of hydrolyzed gelatin and gelatin, and the same compositions for use in methods comprising administering a low dose of the compositions useful in prophylaxis and treatment of hair loss in humans and shedding in animals. The compositions provide a palatable, malleable and well-tolerated product.

### FIELD OF THE INVENTION

The present invention provides compositions and methods useful as prophylaxis and treatment of hair loss in humans and shedding in animals. The compositions comprise certain concentrations of gelatin and provide a palatable, malleable and well-tolerated edible product.

### BACKGROUND OF THE INVENTION

### Hair Loss

Hair loss in men and women has many genetic and physiological and factors. The most common form of balding is Androgenetic Alopecia, or also known as male pattern baldness, which affects over 50% of Caucasian men. Non-genetic events which can trigger hair loss include pregnancy and childbirth; menopause; severe emotional stress; rapid or profound weight loss; malnutrition; medical intervention including surgery and/or radiation; illness; chemotherapy; and hair abuse. Several dietary supplements and topical formulations are commercially available for treatment of hair loss and the promotion of hair growth. None of the so-called “hair growth stimulants” have proven to be very efficacious.

### Gelatin

Gelatin is extracted from by-products of the meat industry including the skin, connective tissue and bones of animals, and comprises primarily proteins. Gelatin is a transparent, essentially odorless and tasteless powder that became a popular consumable in the 1890s when it was first developed and promoted as a commercial product by Charles Knox, founder of the Knox Gelatin Corporation.

Gelatin has long been a key ingredient for “jelled” desserts, frozen drinks, and soft, chewy candies. Gelatin may be prepared from kosher animals, or from fish, to comply with particular dietary laws. Vegetarians may substitute similar jellying agents such as agar, which chemically is not gelatin.

In addition to its use in foodstuff, gelatin, with its dissolvable structure, is also used to manufacture capsules (both hard and “soft-gel”) in the pharmaceutical and neutraceutical industries. It also has industrial and biomedical applications including as an ingredient in film coatings and medical devices such as artificial heart valves, matrices and implants.

### Health Benefits of Gelatin

Although gelatin has been publicized for decades as a good source of protein, there is little hard scientific evidence to support the statements made for gelatin products. For example, gelatin contains protein, but in an incomplete form for mammalian nutrition.

Gelatin has been promoted as a food supplement to improve hair and nail appearance and growth. In fact, the body already manufactures abundant amounts of the amino acids that gelatin provides, and no solid experimental evidence has proven whether extra supplies of these nutrients will help build or maintain healthy hair and nails.

U.S. Pat. No. 4,749,684 teaches a method of promoting linear hair growth comprising administering to a patient not less than 100 mg/kg, or 8-16 grams daily, of gelatin for at least three months. That patent discloses that lower dosages of gelatin are not sufficient to produce an effect on hair growth. Furthermore, the gelatin, when administered in capsular form, was found to be irritating to the subjects, and was therefore provided in yogurt.

US Patent Application No. 20010870424 discloses a method of stimulating hair growth comprising administering intradermally or subdermally, a hydrogel matrix comprising gelatin and a long chain carbohydrate.

U.S. Pat. No. 4,863,950 is directed to a composition for stimulating keratinogenesis and reducing facial and scalp seborrhea formulated as solid spherules comprising about 2 grams “soft” gelatin and about 800 mg L-cysteine. According to the disclosure of this patent, the effect of stimulation of keratinogenesis and reduction of seborrhea is due to the association of “soft” gelatin with cysteine, thereby offering results which are superior to those yielded by presently available formulations which contain only gelatin or only cysteine, or which contain both in different proportions. In fact, the data presented in the '950 patent indicate that animals fed a normal diet with a gelatin supplement for 20 weeks show adverse results, i.e. a slight reduction in the sulphur and cysteine content of the hair keratin.

Different formulations comprising gelatin are taught in the art. For example, U.S. Pat. No. 5,928,664 teaches a consumable elastic gummy glycerylated gelatin matrix admixed with an active ingredient. Gelatin, per se, is neither taught nor suggested as an active ingredient.

U.S. Pat. No. 5,760,094 teaches the incorporation of a small amount of hydrolyzed gelatin into chewable tablets to improve the palatability of medicinals.

U.S. Pat. No. 4,495,206 teaches a solid, high protein dietary food product consisting essentially of a gelatin hydrolysate present in quantities equal to or greater than 50% by weight of the product, and one or more members selected from essential amino acids, preservatives, flavoring agents, coloring agents, texturizers and sweeteners.

U.S. Pat. No. 4,997,671 discloses a chewy, semi-plastic, microbiologically stable dog food, which is a dog snack or biscuit. That dog snack includes 12 to about 30 weight percent, based upon the total weight of the dog food, of gelatin; at least one edible acidulant; at least one edible cereal starch-containing textural agent; at least one edible release agent; at least one edible taste agent; at least one edible sugar; salt; and added water.

That patent teaches gelatin as a textural agent and does not teach gelatin as a nutritive supplement having the effect of preventing or controlling hair loss and excess shedding.

In their Letter to the Editor, Marganti et al J Soc. Cosmet Chem 1982 (33) 95-96, report the use of a combination that comprises 2 grams of gelatin and 0.8 grams of 1-cystine, promoted more intense hair growth and led to substantial increase in the diameter of the fibers together with the increase in the sulfur content of the hair, which implies that the high sulfur protein fraction of hair was affected the most.

There remains a yet unmet need for an edible product useful for preventing hair loss in humans and animals, which is both palatable and well tolerated. The art neither teaches nor suggests such a gelatin based confectionary that provides a dose of gelatin effective for prevention and treatment of mammalian hair loss.

### SUMMARY OF THE INVENTION

The art has provided inconsistent and conflicting reports regarding the usefulness of gelatin in preventing or reducing hair loss and promoting hair and nail growth and health. The inventors of the present invention now show, in contradistinction to the hitherto known art, that a daily low oral dose of gelatin is in the form of hydrolyzed gelatin or a mixture comprising hydrolyzed gelatin and gelatin, and in particular, in the form of a malleable edible composition, is able to reduce or prevent hair loss in mammals. The term “gelatin as an active agent” as used herein throughout the specification and claims is used to denote gelatin that is either in the form of hydrolyzed gelatin or in the form of a mixture comprising hydrolyzed gelatin and gelatin.

The present invention provides a palatable composition comprising gelatin as an active ingredient in an amount sufficient to prevent hair loss in a mammal and to promote hair and nail growth. The composition is tasty and has a surprisingly chewy, soft and non-sticky consistency. Furthermore, the composition which comprises gelatin in its hydrolyzed form is unexpectedly amenable to large-scale formulation and production.

In accordance with a first embodiment of the present invention there is provided an edible composition for use in preventing hair loss in a mammal and comprising gelatin as an active agent, and wherein the gelatin present in the edible composition is in the form of hydrolyzed gelatin or a mixture comprising gelatin and hydrolyzed gelatin.

By yet another embodiment of the invention the edible composition comprises from about 5% to about 50% (w/w) of gelatin as the active agent, out of the overall weight of the composition. In accordance with a preferred embodiment of the present invention, in the case that the active agent, gelatin, is derived from a composition comprising both gelatin and hydrolyzed gelatin, the percentage of gelatin in the non-hydrolyzed form in such a mixture shall preferably not exceed 15% (w/w).

In accordance with the present invention the gelatin may be derived from an animal selected from the group of bovine, porcine, ovine, avian and marine animals. In certain embodiments the gelatin derives from a cow. In specific embodiments the gelatin derives from a fish, and in particular a fish selected from a kosher species of fish.

By yet another embodiment of the present invention, the composition optionally further comprises nutrients useful in prevention of hair loss or promotion of hair growth including one or more members of the group consisting of biotin, iron, copper and zinc ions, para-aminobenzoic acid (PABA), linoleic acid, pantothenic acid, vitamin B6, vitamin B12, vitamin A, vitamin C, vitamin D, folic acid, L-methionine, L-cysteine and selenium. Additional vitamins and minerals, which may aid in the prevention of hair loss or promote hair growth, may be included in the composition.

In accordance with other embodiments the composition further comprises from about 5 to about 250 mcg of L-methionine and/or from about 1 to about 200 mcg of sodium selenide, preferably about 100 mg L-methionine and/or 10 mcg sodium selenide. According to another embodiment, the composition further comprises from about 0.5 to about 20 mg of zinc.

In another aspect the present invention provides an edible composition for promoting hair and nail growth in a mammal provided as a confectionery, preferably as a palatable, chewy candy. In other embodiments the composition is provided as a chewy snack or biscuit or as any other form of animal feed.

In a third aspect the present invention provides a method for reducing or preventing hair loss in a mammal comprising the step of:

administering to the mammal an edible composition comprising gelatin as an active agent, wherein the gelatin is in the form of hydrolyzed gelatin or a mixture comprising gelatin and hydrolyzed gelatin, and wherein the edible composition is administered so that the overall dose of the active agent is in the range of from about 0.5 grams to about 7.0 grams gelatin per day, preferably from about 1.0 to about 3.0 grams gelatin per day. More preferably, the dosing concentration is about 1.5 grams of total gelatin per day.

In one embodiment the mammal is a human. In other embodiments the mammal is a domestic animal selected from a dog, a cat, a primate, a guinea pig, and a rabbit.

The composition may be consumed in one dose or in multiple doses. Likewise, the composition may be consumed one or more times during the day.

In certain embodiments the composition is administered from about 2 to about 8 weeks. Preferably the composition is administered for about 4 to about 6 weeks.

These and other embodiments and objects of the present invention will become apparent in conjunction with the description and claims that follow.

### DETAILED DESCRIPTION OF THE INVENTION

The present invention provides an edible composition comprising from about 5% to about 50% (w/w) hydrolyzed gelatin with or without from about 5% to about 15% (w/w) of gelatin, which is able to reduce or prevent hair loss in humans and shedding in animals. Temporary or chronic non-genetic human hair loss is a common result of certain factors including:
a) Physical stress arising from surgery or rapid weight change
b) Illness including thyroid disorders and pituitary disease, autoimmune disease, scarlet fever or syphilis;
c) Emotional stress and mental illness including anorexia;
d) Medications such as high doses of Vitamin A, certain anticoagulants, gout medications and antidepressants;
e) Hormonal changes including pregnancy, childbirth, birth control pills and devices, menopause;
f) Anti-cancer therapy including chemotherapy and radiation;
g) Hair abuse such as bleaching, permanents, tight braids and pony tails, tight wigs, and tight hats.

The compositions and methods are useful for the prophylaxis and treatment of hair loss in humans and in animals. The compositions are intended as human and veterinary compositions for administration to humans or animals, respectively. The compositions are useful in treating children experiencing hair loss, including pediatric oncology patients.

The compositions provided by the present invention are capable of enhancing the actual density of hair, as can be determined by the actual number of hairs per area on the scalp; and may increase the keratinization of the hair.

The basic edible formulation comprises gelatin. Other ingredients may include additional nutrients, natural and intensified sweeteners, acidulants, colors and flavors. This formulation can be modified by using alternate sugars, varying gelatin levels and blends, and adding gums and starches to reduce the setting time. One objective is achieving the proper texture, sweetness, flavor release and storage properties.

### DEFINITIONS AND EMBODIMENTS

For convenience and clarity certain terms employed in the specification, examples and claims are described herein.

“Growth of hair” and “hair growth” as used herein refers to the growth of the hair from the hair follicle and does not necessarily refer to linear hair growth. Hair growth is intended to mean that the number of hairs per unit area is increased as a result of treatment with the composition of the present invention, as shown by actual hair counts. Without wishing to be bound to theory, growth of hair may result in increased keratinization.

Likewise, “hair loss” refers to a decrease in the number of hairs per unit area as shown by actual hair counts.

Hair follicles grow in repeated cycles. One cycle can be broken down into three phases. Each hair passes through the phases independent of the neighboring hairs.

Anagen is the growth phase. Approximately 85% of all hairs are in this phase at any one time. The anagen phase can vary from two to six years and hair grows approximately 10 cm per year.

Catagen is also known as the transitional phase. At the end of the anagen phase the hairs enters into a catagen phase which lasts about one or two weeks, during which the hair follicle shrinks to about ⅙ of the normal length. The lower part is destroyed and the dermal papilla breaks away.

Telogen, or the resting phase, follows the catagen phase and normally lasts about 5-6 weeks. During this time the hair does not grow but stays attached to the follicle while the dermal papilla stays in a resting phase below. Approximately 10-15 percent of all hairs are in this phase at any one time. At the end of the telogen phase the hair follicle re-enters the anagen phase. The dermal papilla and the base of the follicle join together again and a new hair begins to form. If the old hair has not already been shed the new hair pushes the old one out and the growth cycle starts all over again.

“Gelatin” as used herein refers to a heterogeneous mixture of water-soluble proteins of high average molecular weight derived from collagen by hydrolytic action. Gelatin may be extracted from animal products by acid or base extraction. The difference between the two forms of extracted gelatin is in the isoelectric point of the resultant, soluble polymer. The type and degree of extraction leads to various grades of gelatin. Acid hydrolysis of the collagen leads to Type A gelatin; similarly base hydrolysis leads to Type B gelatin.

Another form of gelatin is the “hydrolyzed gelatin”, which is derived from the standard gelatin by an additional hydrolysis step. Generally molecular weights of the hydrolyzed gelatins are in the 100,000 Dalton range.

Gelatin derives from animal origin, for example from bovine, porcine, ovine, avian or marine sources. In certain embodiments the gelatin derives from fish, preferably a kosher fish including cod, haddock and pollock. The use of gelatin derived from fish obviates the health risks that may be associated with gelatin derived from bovine or ovine sources, but may cause in rare cases allergy to those sensitive to fish products.

Mixtures of different types of gelatin are operable.

The preparation of the composition requires considerable expertise in order to provide a product that is chewable and pleasing to children and adults without being sticky and difficult to chew. Certain parameters must be adhered to.

Gel strength of the gelatin is rated by the bloom strength, and measured on a bloom gelometer, known to those having skill in the art. “Bloom strength” or “bloom” is defined as the weight (measured in grams) required to produce a depression of 4 mm depth in a surface gel by a plate of defined shape and size. The typical strengths of gelatins used for chewy “gummi” candies (such as a “Gummi Bear”) is 175 to 250 grams, or bloom.

Without wishing to be bound to theory, gel strength is important in attaining and retaining the gummi structure, because high temperature and acidic conditions degrade gelatins. Higher gel strengths are less susceptible to this degradation. During processing, the temperature must be kept high to prevent gelling in the system and to allow dispensing of the material.

Gelatin is strongly hydrophilic, absorbing up to ten times its weight of water and forming reversible gels of high strength and viscosity. A thermo reversible gelatin is preferred to allow processing and molding without clogging the production system and dispensing the material in the production lines.

The gelatin used in the compositions of the present invention may be selected from a wide variety of gelatins having a bloom value of about 100-375. A 100-250 Bloom strength of gelatin is preferred, although not critical to the practice of this invention.

Gelatin or hydrolyzed gelatin is employed in the composition of the present invention. Preferably, the composition comprises about 5% to about 15% (w/w) gelatin or about 5% to about 50% (w/w) hydrolyzed gelatin.

“Confectionery” as used herein refers to a palatable, malleable unit that is chewed comfortably, without harming the teeth and jaws, while releasing the nutrients of the gelatin. In certain embodiments the palatable unit is a chewy candy or candy bar that can be chewed to completion.

A dosing regimen is about 1 palatable unit to about palatable units per day. The composition may be consumed in one dose or in multiple doses. Likewise, the composition may be consumed one or more times throughout a 24-hour period.

Increased hair density can be observed within about 2 weeks of administering gelatin to a subject, but it is preferable that the treatment continues about 4 to 6 weeks.

Water comprises a part of the inventive system in an amount from about 5% to about 20% by weight, more preferably about 10 to 15%.

The term “additive” refers to an ingredient, which can be selected from one or more nutrients, flavoring agents, colorants, sweeteners such as isomaltitol and syrup maltitol, stabilizers, humectants, fillers, emulsifiers, thickeners and mixtures thereof. Any one or more of the foregoing additional food grade additives may be admixed with the gelatin matrix so as to comprise from about 5% to about 80% by weight of the composition.

The term “nutrient” refers to a vitamin, a mineral, an amino acid and other chemical or biological substances intended for use in the treatment or prevention of hair loss, or the promotion of hair and nail growth and quality. Mixtures of nutrients are operable.

Exemplary nutrients optionally added to the composition of the present invention one or a mixture of biotin, iron, copper and zinc ions, para-aminobenzoic acid (PABA), linoleic acid, pantothenic acid, vitamin B6, vitamin B12, vitamin A, vitamin C, vitamin D, folic acid, L-methionine, L-cysteine and selenium. Additional vitamins and minerals, which may aid in the prevention of hair loss or promote hair growth, may be included in the composition.

Biotin is a water-soluble vitamin, generally classified as a B-complex vitamin. Biotin may be added at a concentration of about 1 mcg to about 100 mcg per unit. In specific embodiments biotin is included at about 5 to about 20 mcg per unit.

L-Methionine: an essential sulphur containing amino acid and may be included at about 5 to about 250 mcg per unit. In specific embodiments L-methionine is included at about 10 to about 30 mcg per unit.

L-Cysteine is an essential sulphur containing amino acid and may be included at about 5 to about 100 mcg per unit. In specific embodiments L-cysteine is included at about 10 to about 30 mcg per unit.

Para-aminobenzoic acid (PABA) is a component of folic acid. Suitable amounts of PABA range from about 1 mg to about 100 mg per unit.

Zinc is an essential trace element that promotes cell proliferation and tissue growth. Zinc may be added as zinc gluconate, zinc sulfate, zinc picolinate or chelated zinc at a concentration of about 0.5 mg to about mg zinc per unit. In certain embodiments zinc is included at about 1 to about 5 mg per unit.

Copper: a trace element that is essential for the formation of hemoglobin. Copper may be added at a concentration of about 0.1 to about 5 mg per unit.

Selenium is a trace element having antioxidant activity. Selenium may be added to the composition as sodium selenide at about 1 mcg to about 200 mcg per unit.

Because zinc, copper and selenium may be toxic to children at high doses, care is taken to provide a product that will be effective, yet non-toxic if consumed in a large quantity.

Flavoring agents may be chosen from natural and/or synthetic flavoring agents. Commonly used flavors include menthol, artificial vanilla, cinnamon derivatives, and various fruit flavors, whether employed individually or in admixture.

An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes citrus oils such as lemon, orange, lime, mandarin and or grapefruit and fruit essences including apple, pear, peach, grape, strawberry, raspberry, cherry, plum, pineapple, apricot and other fruit flavors. Mixtures and derivatives of these materials are contemplated in the present invention.

Flavor oils include spearmint oil, peppermint oil, cinnamon oil, and oil of wintergreen (methylsalicylate). Clove oil, bay oil, anise oil, eucalyptus oil, thyme oil, cedar leaf oil, oil of nutmeg, oil of sage, oil of bitter almonds and cassia oil can also be used.

Flavorings such as aldehydes and esters include cinnamyl acetate, cinnamaldehyde, citral diethylacetal, dihydrocarvyl acetate, eugenyl formate and p-methylamisol. Further examples of aldehyde flavorings include, but are not limited to acetaldehyde (apple); benzaldehyde (cherry, almond), anisic aldehyde (licorice, anise); cinnamic aldehyde (cinnamon); citral, i.e., alpha citral (lemon, lime); ethyl vanillin (vanilla, cream); hellotropine, i.e., piperonal (vanilla, cream); vanillin (vanilla, cream); alpha-amyl cinnamaldehyde (spicy fruity flavors); butyraldehyde (butter, cheese); valcraldehyde (butter, cheese); citronellal; decannal (citrus fruits); aldehyde C-8 (citrus fruits); aldehyde C-9 (citrus fruits); aldehyde C-12 (citrus fruits); 2-ethylbutyraldehyde (berry fruits); tolyl aldehyde (cherry, almond); veratraldehyde (vanilla); 2,6-dimethyl-5-heptenal, i.e., melonal (melon); 2,6-dimethyloctanal (green fruit); and 2-dodecenal (citrus, mandarin), mixtures thereof and the like.

The amount of flavoring agent employed is normally a matter of preference subject to such factors as flavor type, base type and strength desired. In general, amounts up to about 5% by weight of the flavor agents are usable. In certain embodiments the flavor agent is present from about 0.2% to about 0.5% by weight.

The present invention encompasses according to certain embodiments the inclusion of both natural and intensive, also known as artificial, sweeteners. Any sweetener that is generally recognized as safe (GRAS) is suitable in the present invention. The sweeteners may be chosen from the following non-limiting list: saccharide such as sucrose, glucose (corn syrup), dextrose, invert sugar, fructose, and mixtures thereof; saccharin and its various salts such as the sodium salt; dipeptide sweeteners such as aspartame and neotame; plant derived terpenoids and flavenoids including glycyrrhizin and Stevia Rebaudiana (Stevioside); chloro derivatives of sucrose such as sucralose.

For dietary and health reasons it may be desirable to provide a sugarless formulation in which the sweeteners are be replaced with polyhydric alcohols and mixtures of these alcohols, sugar alcohols such as sorbitol, mannitol, xylitol, erythritol and maltitol and acesulfame-K, and sodium and calcium salts thereof.

In certain embodiments of the present invention, a combination of natural and intensive sweeteners is used.

Natural sweetener, i.e. isomaltitol and syrup maltitol, levels can be as high as about 80% weight of total dry solids, but more typically are from about 50% to about 70% weight percent. The high-intensity sweeteners i.e. aspartame, acesulfame K, saccharin, and sucralose are used at lower concentrations, depending on their potency and the level of sweetness desired in the composition.

Other conventional additives which are useful in the gelatin system of the present invention include mineral adjuvants such as calcium carbonate, magnesium carbonate, alumina, aluminum hydroxide, aluminum silicate, talc, tricalcium phosphate and the like; as well as mixtures thereof. These mineral adjuvants may also serve as fillers and texturizing agents.

Emulsifiers such as lecithin, glycerol monostearate, fatty acid monoglycerides, diglycerides and triglycerides, glycerol triacetate, propylene glycol monostearate and mixtures thereof can also be included in the gelatin composition of the present invention.

For colorants, certified colors are typically used to color the composition. Naturally derived colors are also utilized. Blends and combinations of colorants yield satisfactory shades for many applications.

Certain factors are considered in choosing appropriate colorants, including solubility, dietary limitations, stability to pH, light and heat, and cost.

The final form of the composition is not limited to any shape or size which may be chosen. In some embodiments each palatable unit has a volume of about 1 cm³ to about 10 cm³. Preferably the gelatin mixture is shaped by molding, extrusion or similar techniques known in the art.

Molding starches are used to coat candy molds and ensure the release of the cooled product from the molds. Molding starches are typically unmodified starches that contain added oil at about 0.1% weight to allow the mold shape to be formed. Mineral oil or vegetable oils may be used, as they stable and FDA-approved. Products containing vegetable oils must be treated with antioxidants such as TBHQ, BHA or BHT to deter rancidity.

The product may be coated with a polishing agent to provide the individual palatable unit with a bright appearance and color and prevent of stickiness and moisture loss. These agents may be composed of carnauba wax at about 2%, plus mineral oils, vegetable oils with antioxidants, and medium-chain triglycerides.

Consideration is given to the packaging material, as compatibility with the polish oils of the final product needs to be established.

### Veterinary Product

The present invention further provides an edible composition for hair-bearing animals including domestic animals such as a horse and pets such as dogs, cats and rabbits comprising as an active ingredient about 5% to about 15% (w/w) gelatin. When fed to an animal daily in a proper dosage, the composition reduces or eliminates non-seasonal shedding and promotes healthy skin and a glossy coat, without harmful side effects.

Animals, including humans, do not grow hair continuously, but rather in cycles. Each cycle has a growing phase, a transitional phase and a resting phase. These hair cycles are controlled by such factors as nutrition, hormones, state of health, but most importantly, the amount of light and changes in the surrounding temperature. Pets that are kept primarily indoors tend to experience changes in both temperature and light all year round. As a result, these pets tend to shed their hair, sometimes profusely, throughout the year.

The composition is formulated for each type of animal in order to avoid ingredients that are harmful to that particular animal type. The formulation for an animal is similar to humans and may be provided as a biscuit, snack, dry food, bone shaped, chewy strip etc.

For example, U.S. Pat. No. 4,997,671 discloses a chewy, semi-plastic, microbiologically stable dog food, which is a dog snack or biscuit. That dog snack includes 12 to about 30 weight percent, based upon the total weight of the dog food, of gelatin; at least one edible acidulant; at least one edible cereal starch-containing textural agent; at least one edible release agent; at least one edible taste agent; at least one edible sugar; salt; and added water. The dog food is in a compacted form.

### Example

The following example is to be considered merely as illustrative and non-limiting in nature.

### Methods for Testing Hair Growth

The primary method for testing the compositions' effect on hair growth was to measure total hair counts using fiber-optic microscopy and a computer-assisted, manual hair count method.

Secondary measures of efficacy included assessments of hair diameter, as well as patient and investigator global assessments of improvement in hair growth. These were based on photographs of the scalp using both midline and vertex views.

### Example

### The Effect of the Composition on Human Hair Density

Seventy women, recently post partum, experiencing hair loss were given 1.5 grams gelatin powder daily for one month by oral administration. About 70% of the women reported a significant decrease in hair loss and increase in hair density.

Gelatin was given also to children experiencing hair loss. The gelatin was administered orally with or without multi vitamins. Mothers and children easily accepted the gelatin composition as a treatment and the children experienced a decrease in hair loss and an increase in hair density within about one month.

The gelatin may be provided as is or in combination with vitamins and minerals as described hereinabove. The compositions did not cause any discomfort or side effects in either the children or mothers.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1. An edible composition for use in preventing hair loss in a mammal and comprising gelatin as an active agent, and wherein said gelatin is in the form of hydrolyzed gelatin or of a mixture comprising hydrolyzed gelatin and gelatin.

2. An edible composition according to claim 1, wherein gelatin as the active agent comprises from about 5% to about 50% (w/w) of said edible composition.

3. An edible composition according to claim 1, further comprising at least one nutrient being a member selected from the group consisting of biotin, iron, copper and zinc ions, para-aminobenzoic acid (PABA), linoleic acid, pantothenic acid, vitamin B6, vitamin B12, vitamin A, vitamin C, vitamin D, folic acid, L-methionine, L-cysteine, zinc and selenium.

4. An edible composition according to claim 3, wherein said at least one nutrient is from about 5 to about 250 mcg of L-methionine.

5. An edible composition according to claim 2, wherein said at least one nutrient is from about 1 to about 200 mcg of sodium selenide.

6. An edible composition according to claim 3, wherein said at least one nutrient is from about 0.5 to about 20 mg of zinc.

7. A method for reducing or preventing hair loss in a mammal comprising the step of administering to said mammal an edible composition comprising gelatin as an active agent, wherein said gelatin is in the form of hydrolyzed gelatin or a mixture comprising gelatin and hydrolyzed gelatin, and wherein the administering of said edible composition is carried out so that the overall dose of said active agent is in the range of from about 0.5 grams to about 7 grams of total gelatin per day.

8. A method according to claim 7, wherein said total dose is in the range of from about 1.0 grams to about 3.0 grams of total gelatin per day.

9. A method according to claim 7, wherein the step of administering is carried out by consuming multiple doses during the day.

10. A method according to claim 7, wherein the step of administering is carried out for a period of from about 2 to about 8 weeks.
